(19) United States
(12) Patent Application Publication
(10) Pub. No.: US 2023/0116039 A1
(43) Pub. Date: Apr. 13, 2023

(54) DISINFECTION SOLUTION WITH TWO-PART FORMULATION

(71) Applicant: ASP GLOBAL MANUFACTURING GMBH, SCHAFFHAUSEN (CH)

(72) Inventors: Navid OMIDBAKHSH, Mission Viejo, CA (US); Shokouh Sadat FARVID, Irvine, CA (US)

(21) Appl. No.: 17/914,230

(22) PCT Filed: Apr. 14, 2021
(86) PCT No.: PCT/IB2021/053059

§ 371 Date: Sep. 23, 2022

Related U.S. Application Data
(60) Provisional application No. 63/013,958, filed on Apr. 22, 2020.

(51) Int. Cl. A01N 37/16; A01N 25/22; A01N 25/02; A01P 1/00

(52) U.S. Cl. CPC A01N 37/16; A01N 25/22; A01N 25/02; A01P 1/00

(57) ABSTRACT

A two-part formulation for creating a decontamination solution may comprise a first part comprising a peracid (e.g., peracetic acid (PAA), acetic acid, hydrogen peroxide) and a second part comprising a pH adjuster (e.g., a pH modifier, such as ethanolamine or disodium phosphate), in which the first part and the second part are maintained separately from each other. To assist in increasing the stability of the solution after the first part and the second part are mixed with water, including hard water, the first part and the second part should lack any chelants that could reduce the stability of the solution. Stability reducing chelants may include ethylenediaminetetraacetic acid (EDTA), tetrasodium EDTA, methylglycinediacetic acid trisodium salt, diethylenetriaminepentaacetic acid pentasodium salt, sodium citrate, nitrilotriacetic acid trisodium salt, and glutamic acid diacetic acid tetrasodium salt.

### FIELD

The subject matter disclosed herein relates to liquids that may be used to assist in decontaminating medical devices, particularly decontamination liquids, such as disinfection liquids and sterilization liquids, suitable for use in automatic reprocessing systems.

### BACKGROUND

Various medical devices are used in numerous procedures in the medical field. These devices are as varied as the procedures themselves. As such, proper care of these devices is critical for efficiency of application and the proper corresponding treatment of the patient.

After a medical device, such as a heat sensitive flexible endoscope, is used, the medical device is cleaned and decontaminated (i.e., disinfected or sterilized) in order to prepare the medical device for its next use. The cleaning and decontaminating may include attaching the medical device to a re-processing machine, such as an automated endoscope re-processor (AER), using a connector (a tubing, a fitting, etc.). In order to clean and decontaminate the medical device, the AER can, among other things, circulate a liquid through a lumen of the medical device utilizing a liquid pump.

Certain acids, e.g., peroxygen compounds, including peracids, such as peracetic acid (“PAA”), may be used to assist in decontaminating medical devices. However, in a ready-to-use form, e.g., in a solution, typically an aqueous solution, with a pH adjustment, it has a low stability. This is one reason why peroxygen-based based decontaminants, particularly peracid based decontaminants, that are to be used in solution form are provided to healthcare providers in a form that is not ready to use, e.g., as a two-part kit comprising a Part A and a Part B. Part A may include an acetic acid, peroxyacetic acid, hydrogen peroxide, stabilizers, and sulfuric acid. Part B may include a mixture of chelating agents (e.g., Tetrasodium EDTA, Methylglycinediacetic acid (MGDA), L-glutamic acid N,N-diacetic acid (GLDA), Nitrilotriacetic acid (NTA))), corrosion inhibitors (e.g., 1H-Benzotriazole, sodium salicylate), and pH buffer or pH adjuster. Part A and Part B may be mixed with water to create a pH-adjusted, ready-to-use solution for decontamination.

The chelating agents of Part B are compounds that are capable of binding metal ions, including those commonly found in water, e.g., calcium and magnesium ions. These metal ions are commonly understood in the art to be a factor that contributes to the low stability of ready-to-use solutions because they can catalyze decomposition of peroxides. Because chelants may be used to remove calcium and magnesium ions from water, they are understood to increase the stability of ready-to-use PAA solutions as well as improving disinfection and sterilization performance

### SUMMARY OF THE DISCLOSURE

Certain decontamination solutions, particularly those including a peracid, may have a brief period of stability. As such, the ingredients for making these solutions may be provided in a non-solution form. For example, the ingredients may be provided in a two-part formulation that is mixed together to make the solution when it is time to use the solution. A two-part formulation may comprise a first part comprising a peracid (e.g., peracetic acid (PAA), acetic acid, hydrogen peroxide) and a second part comprising a pH adjuster (e.g., a pH modifier, such as ethanolamine or disodium phosphate), in which the first part and the second part are maintained separately from each other (e.g., the first part is maintained in a first container and the second part is maintained in a second container). To assist in increasing the stability of the solution after the first part and the second part are mixed, the first part and the second part should lack any chelants that could reduce the stability of the solution, i.e., “stability reducing chelants.” Stability reducing chelants may include ethylenediaminetetraacetic acid (EDTA), tetrasodium EDTA, methylglycinediacetic acid trisodium salt, diethylenetriaminepentaacetic acid pentasodium salt, sodium citrate, nitrilotriacetic acid trisodium salt, and glutamic acid diacetic acid tetrasodium salt.

However, stability of the first part may be increased by including in the first part an oxidation-resistant stabilizer, which may include certain oxidation-resistant chelants. Similarly, stability of the solution may be increased by including in the first part or the second part an oxidation-resistant stabilizer, which may include certain chelants. For example, oxidation-resistant stabilizers may include chelants from the phosphonate family, such as amino trimethylene phosphonic acid, diethylene triamine penta (methylene phosphonic acid), 2-hydroxyethyl (amino) bis(methylene phosphonic acid), ethylene diamine tetra(methylene phosphonic acid), and 1-hydroxyethylidene-1, 1-diphosphonic acid. The peracid can be selected from any percarboxylic acids comprising, but not limited to, peracetic acid, perlactic acid, percitric acid, peroctanoic acid, perglycolic acid, perpropionic acid, perglutaric acid, and persuccinic acid.

The second part may be a basic aqueous solution made with water and alkaline solutions (here also referred to as pH modifier) such as sodium hydroxide, potassium hydroxide, sodium phosphate or ethanolamines Further, the second part may include other ingredients, such as a surfactant or a solvent (e.g., glycol ether, propylene glycol, or both). The first part, the second part, and water may be combined or mixed to create a ready-to-use decontamination solution. As such, this solution may include PAA and an oxidation-resistant chelant, while lacking any stability reducing chelants. The PAA may be in the solution with a concentration of between about 0.10% and about 0.50%, e.g., about 0.35%. The solution may have a property whereby the PAA concentration decreases by less than about 30% (e.g., less than about 15%, e.g., about 5%) when maintained at about 56° C. for about one hour. Additionally, the solution may have a pH of between about 3 and about 7, e.g., about 5. The water used to create the solution may comprise tap water, which may be hard water having a metal-ion concentration of between about 100 ppm and 400 ppm, e.g., 200 ppm.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

As noted above, ready-to-use decontamination solutions that comprise an acid, such as a peroxygen compound, may have a low stability. This is particularly so where the acid comprises a peracid. The present disclosure is based on a surprising discovery made by the inventors during research efforts concerning prolonging the stability of these solutions. The inventors discovered that some chelating agents (also referred to herein interchangeably as “chelants”) decrease the stability, and thus disinfection and sterilization efficacy, of these solutions. This discovery is contrary to the accepted view that chelants increase the stability of ready-to-use decontamination solutions by removing metal ions from the solution. This accepted view is disclosed in, e.g., International Publication No. WO2016/082897, which describes using chelants in compositions providing an improved shelf life. The accepted view is also embodied in the S40™ Sterilant Concentrate manufactured by Steris, which includes the chelant tetrasodium EDTA.

The inventors performed various studies to confirm their discovery that at least some chelants reduce the stability of pH-adjusted ready-to-use decontamination solutions, even in solutions comprising hard water. In these studies, peracetic acid (PAA) was combined with hard water having a water hardness of 200 parts per million (“ppm”) such that the resulting solution included a 0.35% concentration of PAA. The pH of the solution was adjusted to 5 using a pH modifier. In a first batch of the solution, the pH modifier used was ethanolamine. In a second batch of the solution, the pH modifier used was disodium phosphate. Test specimens from these two batches were created by individually adding chelants thereto. The chelants used in these studies are those used for removing ions (e.g. Calcium and Magnesium) from hard water, and included tetrasodium EDTA, TRILON® M (methylglycinediacetic acid trisodium salt (MGDA-Na3)), TRILON® C (diethylenetriaminepentaacetic acid pentasodium salt (DTPA-Na5)), sodium citrate, and DISSOLVINE® GL (glutamic acid diacetic acid tetrasodium salt (GLDA-Na4)). Test specimens from these two batches were also created that lacked any such chelants. In some instances, test specimens were created for different concentrations of certain of these chelants. Table 1 reflects test specimens made from the first batch of the solution. Table 2 reflects test specimens made from the second batch of the solution. The tables also reflect the concentration of each chelant in each specimen as a percentage. Each specimen was heated at 56° C. for one hour. At the end of the hour, the PAA concentration was determined. The final concentration of PAA is provided in Table 1 and Table 2 alongside the change in the PAA concentration as determined at the end of the hour (“Δ PAA %”).

TABLE 1
Initial concentration of PAA = 0.35%. Water hardness =
200 ppm. pH of PAA solution adjusted with ethanolamine to 5.
Ready-to-use PAA solution exposed to heat at 56° C. for one hour.

| | Chelant | Final PAA | |
|---|---|---|---|
| Chelant included | Concentration | Concentration | |
| in part B | (%) | (%) | Δ PAA % |
| None | 0 | 0.25 | −29 |
| Na4EDTA | 0.1 | 0.21 | −40 |
| Na4EDTA | 0.4 | 0.14 | −60 |
| Dissolvine (liquid) | 0.2 | 0.23 | −34 |
| Dissolvine (liquid) | 0.6 | 0.17 | −51 |
| Trilon M (liquid) | 0.6 | 0.19 | −46 |
| Trilon C (liquid) | 0.2 | 0.22 | −37 |
| sodium citrate | 0.4 | 0.23 | −34 |

TABLE 2
Initial concentration of PAA = 0.35%. Water hardness =
200 ppm. pH of PAA solution adjusted with disodium phosphate to
5. Ready-to-use PAA solution exposed to heat at 56° C. for one hour.

| | Chelant | Final PAA | |
|---|---|---|---|
| Chelant included | Concentration | Concentration | |
| in part B | (%) | (%) | Δ PAA % |
| None | 0 | 0.32 | −9 |
| Na4EDTA | 0.1 | 0.28 | −20 |
| Na4EDTA | 0.4 | 0.18 | −49 |
| Trilon M (liquid) | 0.2 | 0.3 | −14 |
| Trilon M (liquid) | 0.6 | 0.26 | −26 |
| Dissolvine (liquid) | 0.2 | 0.29 | −17 |
| Dissolvine (liquid) | 0.6 | 0.25 | −29 |
| Trilon C (liquid) | 0.2 | 0.26 | −26 |
| sodium citrate | 0.4 | 0.26 | −26 |

As reflected in Table 1 and Table 2, the PAA concentration in each specimen of the solution decreased from the starting concentration of 0.35%. The decrease was least for the two specimens that lacked any chelant. Specifically, when the pH was adjusted using ethanolamine, the PAA concentration in the specimen lacking any chelants decreased by about 29%, whereas the PAA concentration decreased by up to 60% when chelants were included. Further, when the pH was adjusted using disodium phosphate, the PAA concentration in the specimen lacking any chelants decreased by about 9%, whereas, the PAA concentration decreased by up to 49% when chelants were included. This discovery was surprising in light of the accepted view that chelants increase the stability of ready-to-use solutions by removing metal ions from the solution. Yet even in specimens of a pH-adjusted, ready-to-use solution made from hard water, the data collected by the inventors as provided in Table 1 and Table 2 do not support the accepted view.

Practical applications of the inventors' discovery include, e.g., manufacturing products and pH adjusting solutions that lack any chelants that would reduce the stability of a ready-to-use solution as determined by a test, such as the test described above. Any chelants that may be shown to reduce the stability of a solution containing peroxygen compounds are referred to collectively herein as “stability reducing chelants.” Examples of such stability reducing chelants include those described above and others, such as: a) ethylenediaminetetraacetic acid (EDTA), b) tetrasodium EDTA, TRILON® M (methylglycinediacetic acid trisodium salt (MGDA-Na3)), c) TRILON® C (diethylenetriaminepentaacetic acid pentasodium salt (DTPA-Na5)), d) sodium citrate, e) NTA (nitrilotriacetic acid trisodium salt), and f) DISSOLVINE® GL (glutamic acid diacetic acid tetrasodium salt (GLDA-Na4)).

In one such application, a two-part formulation for decontamination (such as disinfection and sterilization), comprising a Part A (i.e., a first part) and a Part B (i.e., a second part), may be provided. Part A may be comprised of a peracid comprising PAA, acetic acid, hydrogen peroxide, stabilizer(s), or any combination thereof. The PAA in part A may be formed by combining the hydrogen peroxide and acetic acid. Furthermore, Part A may contain stabilizers that are resistant to oxidation, which may include certain chelants. For example, the stabilizers may include chelating agents from the phosphonate family, because these chelating agents do not react with peroxygen compounds, such as PAA, and thus, such chelating agents increase the stability of the PAA in Part A. Specific examples of such oxidation-reducing chelants include, but are not limited to, e.g., chelating agents from the phosphonic acid/phosphonate family such as: a) amino trimethylene phosphonic acid, b) diethylene triamine penta (methylene phosphonic acid), c) 2-hydroxyethyl (amino) bis(methylene phosphonic acid), d) ethylene diamine tetra(methylene phosphonic acid), and e) 1-hydroxyethylidene-1, 1-diphosphonic acid. Additionally or alternatively, phosphonic acids marketed under the trade name DEQUEST® by Italmatch Chemicals may be included in Part A. Examples include DEQUEST® 2000, 2010, 2060, 2070, and 2090.

Part B may be comprised of a pH adjuster, such as a pH buffer or pH modifier (e.g., ethanolamine, disodium phosphate, sodium hydroxide, potassium hydroxide), to achieve a desired pH of the ready-to-use solution. Part B may additionally include surfactants, preferably low foaming surfactants (e.g. fatty alcohol ethylene oxide/propylene oxide copolymer derivative, polyoxyethylene-polyoxypropylene block copolymer), solvents (e.g., glycol ether and propylene glycol), and corrosion inhibitors (e.g. 1H- Benzotriazole, sodium salicylate).

Based on the inventors' discovery, Part B should not contain any stability reducing chelants. Preferably, Part B lacks any chelants. Nonetheless, chelants that are not stability reducing chelants, such as those that may be included in Part A as described above, may be included in Part B. However, inclusion of any such chelants in Part B could require that additional buffers or other chemicals be added to Part B such that a correct pH of the ready-to-use solution would be achieved. The addition of these buffers or other chemicals could potentially increase the cytotocitiy effect of the solution on the instrument (e.g. flexible endoscope) more than if Part B simply lacked any chelants.

Part A and Part B should be maintained, provided, and stored separately from each other, e.g., in an unmixed state, such as in a kit comprising separate containers or in separate compartments of a single container. That is, Part A may be contained in a first container and Part B may be contained in a second container. Part A and Part B may be combined with another liquid, such as water, including hard tap water (having metal ions less than about 100 ppm to about 400 ppm, e.g., 200 ppm), to create a pH-adjusted, ready-to-use solution, which may have a pH between about 3 and about 7, e.g., between about 3.7 and about 4.3, or about 5. The ready-to-use solution may include PAA at a concentration of between about 0.10% to about 0.5%, e.g., about 0.35%. Based on the data in Table 1 and Table 2, the pH-adjusted ready-to-use solution exhibits a decrease in PAA concentration, after being maintained at about 56° C. for about one hour, of about less than 30%, e.g., less than about 15%, such as about 5%.

Because tap water is abundant, it can be used for diluting peracid solution. In case the hardness of the tap water is excessive, it may be required to treat it with a water softer to reduce water hardness before entering the system. Such a treatment should nonetheless be less costly and less complicated than using deionized or sterile water. Additionally, the tap water may be passed through a filter or a cascade of filters (e.g., 0.1 to 0.4 microns) to capture particles and microorganisms (e.g. bacteria). Accordingly, the use of a PAA solution lacking any of the stability reducing chelants not only provides improved stability, but also should lower related costs associated with using water from sources besides the tap to create ready-to-use solutions.

As such, a Part A and a Part B may be provided in separate containers, e.g., cups, to be mixed by a healthcare provider with, e.g., deionized water or hard tap water. Table 3 reflects an exemplary formulation of Part A, in which the peracetic acid was formed by allowing the acetic acid and hydrogen peroxide to react and reach equilibrium. Table 4 reflects three exemplary formulations of part B. Table 5 reflects properties of three ready-to-use solutions that result from mixing the Part A of Table 3 respectively with the three different formulations of Part Bs, and different masses thereof, of Table 4, with deionized water. Table 5 also reflects the pH of the solutions right after mixing, the PAA percentage right after mixing, the PAA percentage after maintaining the solution at about 56° C. during about sixty minutes after mixing, and the PAA percentage loss.

TABLE 3
Exemplary Part A Formulations

| | Formulation | Formulation | Formulation |
|---|---|---|---|
| | 1A | 2A | 3A |
| Ingredient | w/w % | w/w % | w/w % |
| Deionized water | Qs to 100 | 8.5 | 18.3 |
| Acetic acid | <40 | 30 | 47 |
| Hydrogen peroxide (50%) | <40 | 60 | 34 |
| Oxidation-resistant | <5 | 1.5 | 0.7 |
| stabilizer | | | |
| Peracetic acid generated | 15 | 15 | 15 |
| from the reaction of | | | |
| peroxide and acetic acid | | | |

TABLE 4
Exemplary Part B formulations.

| | | Formulation | Formulation | Formulation |
|---|---|---|---|---|
| | | 1B | 2B | 3B |
| Ingredient | Supplier | w/w % | w/w % | w/w % |
| Deionized Water | NA | 54.5 | 70 | 90 |
| propylene glycol | Ward's | 10 | 10 | 0 |
| | Science | | | |
| Sodium xylene | Sigma | 15 | 10 | 0 |
| sulfonate solution | Aldrich | | | |
| Bioterge PAS-8S | Stepan | 0.5 | 0 | 0 |
| NaOH | Sigma | 0 | 10 | 10 |
| | Aldrich | | | |
| Ethanolamine | Sigma | 20 | 0 | 0 |
| | Aldrich | | | |

TABLE 5
Part A + Part B + Hard Water formulations. Part A and part B were
mixed in 200 PPM hard water, and then preheated to 56° C.

| | | | | PAA |
|---|---|---|---|---|
| | pH | PAA % | PAA % | percentage |
| | (0 min) | (0 min) | (60 min) | loss |
| Solution 1 | 4.34 | 0.4 | 0.33 | 18 |
| Part A, Formulation 1A: 2.67 | | | | |
| (g) | | | | |
| Part B, Formulation 1B: 1.48 | | | | |
| (g) | | | | |
| Solution 2 | 4.37 | 0.4 | 0.38 | 5 |
| Part A, Formulation 1A 2.67 | | | | |
| (g) | | | | |
| Part B, Formulation 2B: 1.92 | | | | |
| (g) | | | | |
| Solution 3 | 4.78 | 0.35 | 0.33 | 6 |
| Part A, Formulation 1A: 2.33 | | | | |
| (g) | | | | |
| Part B, Formulation 3B: 2.3 | | | | |
| (g) | | | | |

By virtue of the embodiments illustrated and described herein, Applicant has devised a method and variations thereof for preparing pH-adjusted, ready-to-use PAA solutions using water, including hard tap water. The method and its variations comprise a step of mixing any of the Parts A and Parts B described herein with a volume of water. The water may be purified water, deionized water, or tap water. In those variations where tap water is used, the tap water may have a hardness of between about 100 ppm and about 400 ppm, e.g., 200 ppm. Where the ready-to-use solution comprises PAA, the solution exhibits a property whereby, after being maintained at about 56° C. for about one hour, the PAA concentration decreases by less than 30%, e.g., less than about 15%, such as about 5%.

Accordingly, the ready-to-use solution may be used to decontaminate a medical device according to the following method and variations. First, a Part A and a Part B may be received by a user, e.g., a healthcare provider or an employee thereof. In some variations of the method, a first container containing Part A and a second container containing Part B may be received by the user. In other variations, a single container comprising separate compartments, one containing Part A and one containing Part B, may be received by the user. Second, in all of these variations, the user may open the container or containers. Third, as described in the preceding paragraph, the user may prepare a ready-to-use solution by combining the Part A, the Part B, and water, including hard water. In those variations where Part A comprises PAA, the ready-to-use PAA solution exhibits a property whereby, after being maintained at about 56° C. for about one hour, the PAA concentration in the solution decreases by less than 30%, e.g., less than about 15% or less than about 10%, such as about 5%, or less. Fourth, the user may fill a reservoir of a decontamination system. e.g., an automatic endoscope reprocessor, with the ready-to-use solution PAA solution or with part A, part B and water to make the ready-to-use PAA solution. Fifth, the user may position a medical device, such as an endoscope, in a decontamination area, e.g., basin, of the decontamination system. Sixth, the user may activate the system. Finally, the user may remove the medical device in a decontaminated state from the system.

Any of the examples or embodiments described herein may include various other features in addition to or in lieu of those described above. The teachings, expressions, embodiments, examples, etc., described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

1. A two-part formulation, comprising:
a first part comprising a peracid; and
a second part comprising a pH adjuster,
in which the first part and the second part are maintained separately from each other, and
in which the second part lacks any stability reducing chelants.

2. The two-part formulation of claim 1, in which the stability reducing chelants include ethylenediaminetetraacetic acid (EDTA), tetrasodium EDTA, methylglycinediacetic acid trisodium salt, diethylenetriaminepentaacetic acid pentasodium salt, sodium citrate, nitrilotriacetic acid trisodium salt, and glutamic acid diacetic acid tetrasodium salt.

3. The two-part formulation of claim 1 or 2, in which the first part comprises an oxidation-resistant stabilizer.

4. The two-part formulation of claim 3, in which the oxidation-resistant stabilizer comprises an oxidation-resistant chelant.

5. The two-part formulation of claim 4, in which the oxidation-resistant chelant comprises an oxidation-resistant chelant of the phosphonate family.

6. The two-part formulation of claim 4, in which the oxidation-resistant chelant is selected from the group consisting of amino trimethylene phosphonic acid, diethylene triamine penta (methylene phosphonic acid), 2-hydroxyethyl (amino) bis(methylene phosphonic acid), ethylene diamine tetra(methylene phosphonic acid), and 1-hydroxyethylidene-1, 1-diphosphonic acid.

7. The two-part formulation of claim 5, in which the peracid comprises peracetic acid (PAA).

8. The two-part formulation of claim 5, in which the pH adjuster comprises a pH modifier.

9. (canceled)

10. (canceled)

11. (canceled)

12. (canceled)

13. (canceled)

14. (canceled)

15. (canceled)

16. A ready-to-use decontamination solution, comprising:
a peracid;
a pH adjuster; and
water,
in which the ready-to-use decontamination solution lacks any stability reducing chelants.

17. The ready-to-use decontamination solution of claim 16, in which the stability reducing chelants include ethylenediaminetetraacetic acid (EDTA), tetrasodium EDTA, methylglycinediacetic acid trisodium salt, diethylenetriaminepentaacetic acid pentasodium salt, sodium citrate, nitrilotriacetic acid trisodium salt, and glutamic acid diacetic acid tetrasodium salt.

18. The ready-to-use decontamination solution of claim 17, further comprising an oxidation-resistant stabilizer.

19. The ready-to-use decontamination solution of claim 18, in which the oxidation-resistant stabilizer comprises an oxidation-resistant chelant.

20. The ready-to-use decontamination solution of claim 19, in which the oxidation-resistant chelant comprises an oxidation-resistant chelant from the phosphonate family.

21. The ready-to-use decontamination solution of claim 19, in which the oxidation-resistant chelant is selected from the group consisting of amino trimethylene phosphonic acid, diethylene triamine penta (methylene phosphonic acid), 2-hydroxyethyl (amino) bis(methylene phosphonic acid), ethylene diamine tetra(methylene phosphonic acid), and 1-hydroxyethylidene-1, 1-diphosphonic acid).

22. The ready-to-use decontamination solution of claim 20, in which the peracid comprises peracetic acid (PAA).

23. The ready-to-use decontamination solution of claim 22, in which the ready-to-use decontamination solution includes the PAA at a PAA concentration of between about 0.10% and about 0.50%, and in which a property of the ready-to-use decontamination solution is that the PAA concentration decreases by less than about 30% when maintained at about 56° C. for about one hour.

24. (canceled)

25. (canceled)

26. The ready-to-use decontamination solution of claim 23, in which the property of the ready-to-use decontamination solution is that the PAA concentration decreases by less than about 15% when maintained at about 56° C. for about one hour.

27. The ready-to-use decontamination solution of claim 26, in which the property of the ready-to-use decontamination solution is that the PAA concentration decreases by about 5% when maintained at about 56° C. for about one hour.

28. (canceled)

29. (canceled)

30. (canceled)

31. (canceled)

32. The ready-to-use decontamination solution of claim 23, in which the water comprises hard water having a metal-ion concentration of between about 100 ppm and 400 ppm.

33. (canceled)

34. (canceled)

35. A method of preparing a decontamination solution, comprising:
receiving a two-part formulation comprising
a first part comprising a peracid, and
a second part comprising a pH adjuster,
in which the first part and the second part are maintained separately from each other, and
in which the second part lacks any stability reducing chelants.

36. The method of claim 35, in which the stability reducing chelants include ethylenediaminetetraacetic acid (EDTA), tetrasodium EDTA, methylglycinediacetic acid trisodium salt, diethylenetriaminepentaacetic acid pentasodium salt, sodium citrate, nitrilotriacetic acid trisodium salt, and glutamic acid diacetic acid tetrasodium salt.

37. The method of claim 36, in which the first part comprises an oxidation-resistant stabilizer.

38. The method of claim 37, in which the oxidation resistant stabilizer comprises and oxidation-resistant chelant.

39. The method of claim 38, in which the oxidation-resistant chelant comprises an oxidation-resistant chelant from the phosphonate family.

40. The method of claim 38, in which the oxidation-resistant chelant is selected from the group consisting of amino trimethylene phosphonic acid, diethylene triamine penta (methylene phosphonic acid), 2-hydroxyethyl (amino) bis(methylene phosphonic acid), ethylene diamine tetra(methylene phosphonic acid), and 1-hydroxyethylidene-1, 1-diphosphonic acid.

41. The method of claim 39, in which the peracid comprises peracetic acid (PAA).

42. The method of claim 41, in which the ready-to-use decontamination solution includes the PAA at a PAA concentration of between about 0.10% and about 0.50%, and in which a property of the ready-to-use decontamination is that the PAA concentration decreases by less than about 30% when maintained at about 56° C. for about one hour.

43. (canceled)

44. (canceled)

45. The method of claim 42, in which the property of the ready-to-use decontamination is that the PAA concentration decreases by less than about 15% when maintained at about 56° C. for about one hour.

46. (canceled)

47. (canceled)

48. (canceled)

49. (canceled)

50. (canceled)

51. (canceled)

52. (canceled)

53. The method of claim 42, further comprising combining the first part and the second part with a volume of water to create the ready-to-use decontamination solution.

54. (canceled)

55. (canceled)

56. (canceled)

57. (canceled)

58. The method of claim 53, in which the volume of water comprises hard water having a metal-ion concentration of between about 100 ppm and 400 ppm.

59. (canceled)

60. (canceled)

61. (canceled)
